(19) United States
(12) Patent Application Publication
(10) Pub. No.: US 2024/0156586 A1
(43) Pub. Date: May 16, 2024

(54) Graft Tensioning System with Modular Engagement Element

(71) Applicant: Acuitive Technologies, Inc., Allendale, NJ (US)

(72) Inventors: Douglas William Gabel, Ho-Ho-Kus, NJ (US); Michael P. McCarthy, Ho-Ho-Kus, NJ (US); Rui J. Ferreira, Livingston, NJ (US)

(73) Assignee: Acuitive Technologies, Inc., Allendale, NJ (US)

(21) Appl. No.: 18/279,991

(22) PCT Filed: Feb. 24, 2022
(86) PCT No.: PCT/US2022/017724

§ 371 Date: Sep. 1, 2023

Related U.S. Application Data
(60) Provisional application No. 63/157,407, filed on Mar. 5, 2021.

(51) Int. Cl. A61F 2/08

(52) U.S. Cl. CPC A61F 2/0811; A61F 2220/0008

(57) ABSTRACT

Graft tensioning systems and associated engagement assemblies are provided. The engagement assemblies include at least one engagement element or foot that is configured to engage with tissue adjacent or opposed to an implant-receiving opening. The engagement element(s) support stable interaction between the graft tensioning system and the anatomical target region, while reducing the potential for tissue bruising/trauma/contusions. The engagement elements may be interchangeably mounted relative to the graft tensioning device, e.g., intraoperatively, to permit a surgeon to select an engagement element offering desired properties for the specific procedure and/or anatomical properties of the patient.

### CROSS-REFERENCE TO RELATED APPLICATION

The present application claims priority benefit to a U.S. provisional application entitled “Graft Tensioning System with Modular Engagement Element,” which was filed on Mar. 5, 2021, and assigned Ser. No. 63/157,407. The entire content of the foregoing provisional application is incorporated herein by reference.

### BACKGROUND

### 1. Technical Field

The present disclosure relates to a graft tensioning system that includes at least one engagement element or foot that is configured to engage with tissue adjacent to an implant-receiving opening. The disclosed engagement element(s) support stable interaction between the graft tensioning system and the anatomical target region, while reducing the potential for tissue bruising/trauma/contusions. The disclosed engagement elements may be interchangeably mounted relative to the graft tensioning device, e.g., intraoperatively, to permit a surgeon to select an engagement element offering desired properties for the specific procedure and/or anatomical properties of the patient.

### 2. Background Art

Graft tensioning devices are known in the art, and such devices find application in a range of surgical procedures, particularly in connection with joint-related procedures. For example, graft tensioning devices are routinely used in repairs of the anterior cruciate ligament (ACL) in which a soft tissue graft is attached at both ends through a hole drilled through the two bones that make up the knee joint: the femur and the tibia. Graft tension in ACL reconstruction is a factor in favorable clinical outcomes in ACL reconstruction procedures. See, e.g., Markolf et al., “Biomechanical Consequences of Replacement of the Anterior Cruciate Ligament With a Patellar Ligament Allograft. Part Two: Forces in the Graft Compared with Forces in the Intact Ligament,” J. Bone Joint Surg. Am., 78: 11, 1728-34 (November 1996); Tohyama et al., “Significance of Graft Tension in Anterior Cruciate Ligament Reconstruction. Basic background and clinical outcome,” Knee Surg. Sports Traumatol. Arthroscopy, 6 Suppl. 1, S30-7 (1998); Andersen et al., “Review on Tension in the Natural and Reconstructed Anterior Cruciate Ligament,” Knee Surg. Sports Traumatol. Arthroscopy, 2:4, 192-202 (1994); Yasuda et al., “Effects of Initial Graft Tension on Clinical Outcome After Anterior Cruciate Ligament Reconstruction. Autogenous Doubled Hamstring Tendons Connected in Series of Polyester Tapes,” Am. J. Sports Med., 25: 1, 99-106 (January 1997); Hamner et al., “Hamstring Tendon Grafts for Reconstruction of the Anterior Cruciate Ligament: Biomechanical Evaluation of the Use of Multiple Strands and Tensioning Techniques,” J. Bone Joint Surg. Am., 81:4, 549-57 (April 1999).

The patent literature also reflects work in developing beneficial tensioning devices/systems. See, e.g., U.S. Pat. Nos. 4,712,542; 5,037,426; U.S. Pat. No. Re 34,762; U.S. Pat. Nos. 5,713,897; 5,507,750; and 5,562,668.

Ligament and tendon repair procedures extend beyond knee-related procedures. For example, ligament and tendon repair procedures are routinely undertaken with respect to other anatomical regions, e.g., the foot and ankle, the shoulder and rotator cuff, the elbow, and the wrist and hand. In knee-related procedures, e.g., ACL repairs, it is frequently desirable to stabilize a graft tensioning device relative to the anatomical region of the patient. Such stabilization functionality is ideally accomplished with minimal bruising/trauma to the soft tissue surrounding the ligament/tendon introduction site, while permitting efficient access thereto for purposes of the repair procedure. Graft tensioning devices are generally not available and/or employed for use in anatomical regions other than the knee because tissue engagement requirements of other anatomical regions have not been effectively addressed.

Despite efforts to date, a need remains for devices and systems for use in ligament/tendon repair that provide effective stabilization while minimizing the potential for bruising/trauma to soft tissue surrounding the ligament/tendon introduction site. A need further remains for devices/systems that facilitate clinical flexibility in ligament/tendon procedures, whereby a surgeon/practitioner is able to easily/reliably select a desired stabilization element based on clinical variables and/or surgeon/practitioner preference. These and other needs are satisfied by the devices, systems and associated methods disclosed herein.

### SUMMARY

The present disclosure provides stabilization devices, systems and methods for use in connection with graft tensioning procedures, wherein the stabilization functionality is effectuated by at least one engagement element or foot that is configured to engage with either the bone or tissue adjacent or opposed to an implant-receiving opening depending on the procedure and/or surgeon's preferred technique. The disclosed engagement element(s) support stable interaction between the graft tensioning system and the anatomical target region, while reducing the potential for tissue bruising/trauma/contusions.

The disclosed engagement elements may be interchangeably mounted relative to the graft tensioning device, e.g., intraoperatively, to permit a surgeon/practitioner to select an engagement element offering desired properties for the specific procedure and anatomical properties of the patient. The disclosed engagement elements may include an attachment mechanism that facilitates ease of attachment/detachment from a graft tensioning device. The graft tensioning device may include one or more ancillary functionalities, e.g., a tensioning gauge and/or a guide channel for interaction with and tensioning of an implant.

In exemplary clinical implementations of the disclosed graft tensioning device, the device may be positioned by the surgeon on the side that is opposite the side on which the implant is inserted or alternatively may be placed on the same side on which the implant is inserted. Thus, in such clinical implementations, the disclosed graft tensioning device is effective to interact with an implant, e.g., a graft, that has been inserted to an anatomical channel on an opposite side of the patient and pull/draw the implant/graft (and/or a suture associated with the implant/graft) into the device, applying a desired level of tension to the implant/graft before fixation in the implant site.

In further embodiments of the present disclosure, the disclosed graft tensioning device may be secured relative to an engagement assembly that is particularly suited to other types of clinical procedures, e.g., ACL replacement procedures. The engagement assembly may define spaced guide arms that facilitate controlled routing of sutures from the implant region to a suture retention structure associated with the graft tensioning device. The suture retention structure may be rotatably mounted relative to the graft tensioning device, thereby permitting the suture retention structure to rotate/swivel in response to the forces exerted by/to the sutures on either side of the central axis of the graft tensioning device. In this way, the forces applied to/experienced by the sutures may be advantageously balanced.

Additional features and functions associated with the disclosed engagement element(s) and graft tensioning system that may be used in conjunction with the disclosed engagement element(s) will be apparent from the description which follows, particularly when read in conjunction with the appended figures.

### BRIEF DESCRIPTION OF FIGURES

To assist those of skill in the art in making and using the disclosed engagement elements and associated graft tensioning systems, reference is made to the accompanying figures, wherein:

FIG. 1 is a side view of an exemplary engagement assembly according to the present disclosure;

FIG. 2 is a perspective view of the engagement assembly of FIG. 1;

FIG. 3 is a perspective view of an exemplary graft tensioning device for use with an engagement assembly of FIGS. 1 and 2;

FIG. 4 is a perspective view of the engagement assembly of FIGS. 1 and 2 detachably mounted with respect to the graft tensioning device of FIG. 3;

FIG. 4A is a side view, partially in section, of the graft tensioning device/engagement assembly of FIG. 4;

FIG. 5 is a side view of the distal portion of the graft tensioning device/engagement assembly of FIG. 4;

FIG. 6 is a bottom view of the engagement assembly of FIGS. 1 and 2;

FIG. 7 is a further view of the engagement assembly of FIGS. 1 and 2 detachably mounted with respect to the graft tensioning device of FIG. 3;

FIG. 8 is a side view of an alternative engagement assembly according to the present disclosure;

FIG. 9 is an isometric view of the alternative engagement assembly of FIG. 8;

FIG. 10 is a side view of a further alternative engagement assembly according to the present disclosure;

FIG. 11 is an isometric view of an additional alternative engagement assembly according to the present disclosure;

FIG. 12 is a side view of the additional alternative engagement assembly of FIG. 11;

FIG. 13 is a side view of a graft tensioning device/engagement assembly of the present disclosure in operative orientation relative to a patient's foot;

FIG. 14 is an isometric view of a further alternative graft tensioning device/engagement assembly according to the present disclosure;

FIG. 15 is an isometric view of another alternative graft tensioning device/engagement assembly according to the present disclosure;

FIG. 16A and FIG. 16B are side views, rotating by approximately 90° relative to each other, of the further alternative graft tensioning device/engagement assembly of FIG. 14;

FIG. 17A and FIG. 17B are side views, rotating by approximately 90° relative to each other, of the alternative graft tensioning device/engagement assembly of FIG. 15;

FIG. 18 is a top view of the further alternative graft tensioning device/engagement assembly of FIG. 14; and

FIGS. 19-23 are images of a prototype version of the alternative graft tensioning device/engagement assembly of FIG. 15 in association with a model of a knee showing the interplay of the graft tensioning device/engagement assembly with suture.

### DESCRIPTION OF EXEMPLARY EMBODIMENTS

The present disclosure provides stabilization devices, systems and methods for use in connection with graft tensioning procedures. The disclosed stabilization devices, systems and methods generally include at least one engagement element or foot that is configured to mount relative to a graft tensioning device. In exemplary embodiments of the present disclosure, the engagement element is associated with an engagement assembly that detachably mounts relative to a graft tensioning device. However, the present disclosure is not limited by or to implementations wherein interaction between the engagement element/engagement assembly and the graft tensioning device is detachable. Rather, the present disclosure expressly encompasses implementations where the engagement element/engagement assembly is integrally formed with the graft tensioning device, i.e., as a unitary assembly.

The disclosed engagement element(s) support stable interaction between the graft tensioning system and the anatomical target region, while reducing the potential for tissue bruising/trauma/contusions. The graft tensioning device may include one or more ancillary functionalities, e.g., a tensioning gauge, guide channel(s) for passage of instrument(s)/implant(s), and the like.

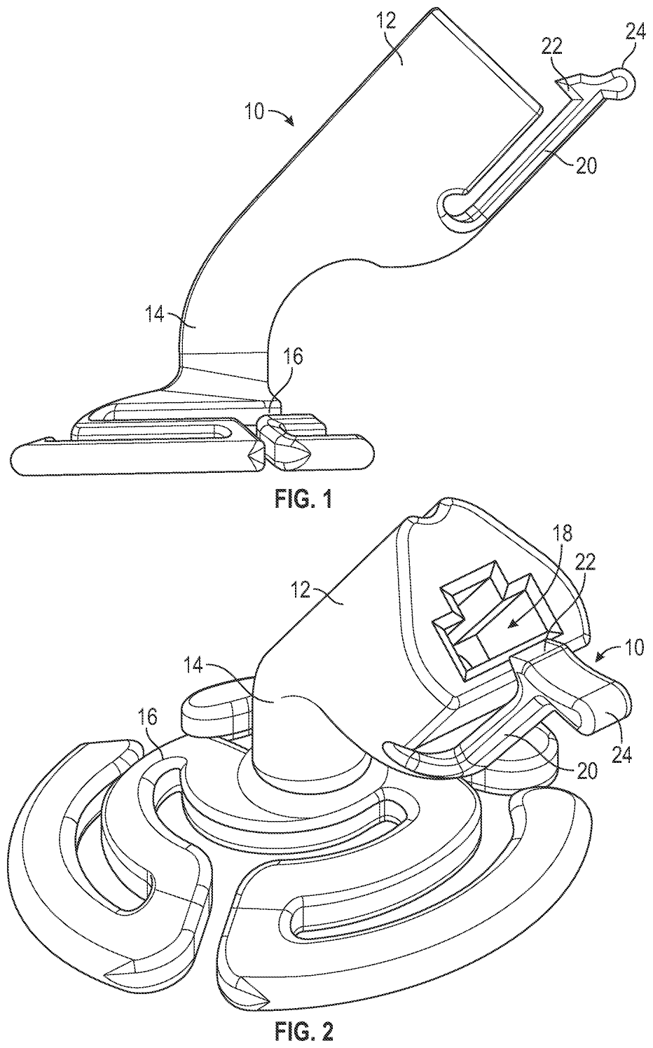

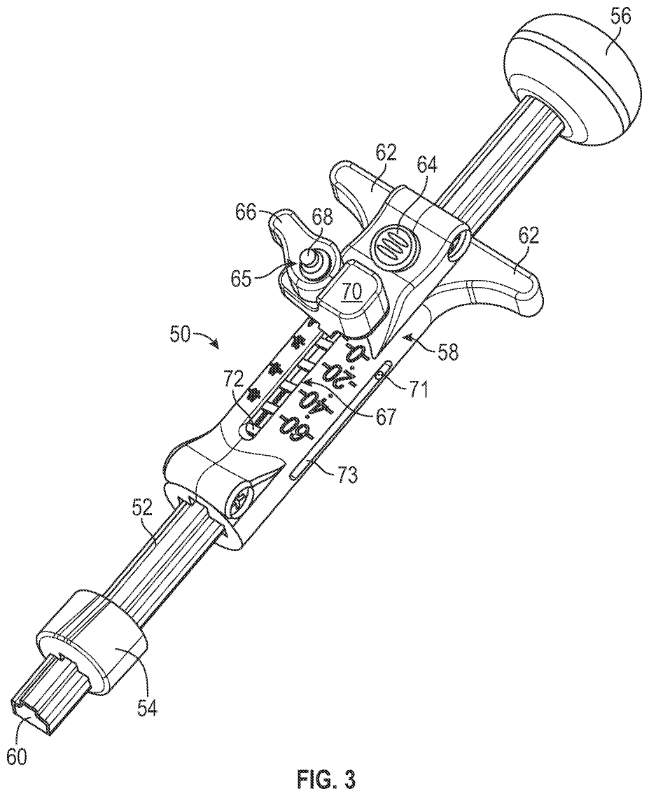

With initial reference to FIGS. 1-2, an exemplary engagement assembly 10 is schematically depicted. Engagement assembly 10 defines a mounting region 12, an intermediate contoured region 14 that extends from the mounting region 12, and an engagement element 16 associated with the distal end of the contoured region 14. The mounting region 12 generally defines a proximally directed mounting feature 18 that is adapted to cooperate with a distally directed mounting feature (see FIG. 3) that engages therewith. In the exemplary embodiment of FIG. 2, mounting feature 18 takes the form of a keyed opening that is configured to interact with a corresponding keyed extension (see, e.g., keyed extension 60 in FIG. 3) associated with a cooperative tensioning device (see, e.g., tensioning device 50 in FIG. 3). The present disclosure is not limited by or to the disclosed mounting feature geometries and/or male/female mounting mechanism. For example, the keyed opening and associated keyed extension may be fabricated with differing cooperative geometries (e.g., oval geometries, elliptical geometries, polygonal geometries, etc.).

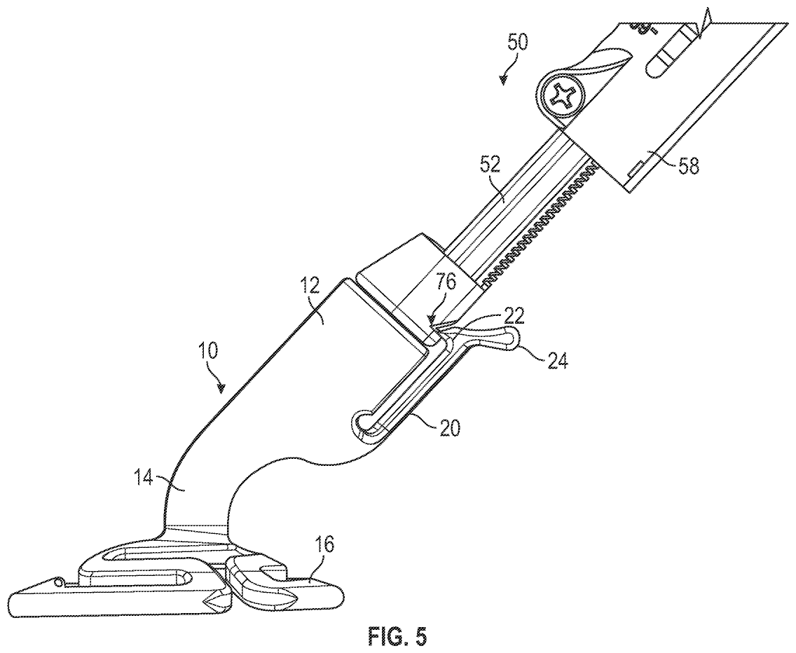

With further reference to FIGS. 1, 2 and 6, engagement assembly 10 includes a deflectable latch arm 20 that extends from mounting region 12 and that defines a latching hook 22 at (or near) a proximal end thereof. Latch arm 20 is adapted to deflect outward relative to mounting region 12 of engagement assembly 10 to permit introduction of a keyed extension (or other mounting feature) associated with a tensioning device to mounting feature 18, and then to flex inwardly such that latching hook 22 engages an associated latching feature associated with the tensioning device (see, e.g., FIG. 5). In exemplary embodiments, latch arm 20 may further define a release nub 24 that facilitates outward deflection of the latch arm 20 relative to mounting region 12 to facilitate detachment of engagement assembly 10 from a tensioning device, i.e., to allow detachment of the engagement assembly 10 from the tensioning device.

The contour region 14 angles relative to the mounting region 12 of engagement assembly 10. In the exemplary embodiment of FIGS. 1 and 2, contour region 14 defines an arcuate transition from mounting region 12 to engagement element 16. However, the present disclosure is not limited by or to a fixed contour as shown with reference to engagement assembly 10. For example, the transition from mounting region 12 to engagement element 16 may take the form of a mechanical joint that may permit different angular orientations between mounting region 12 and engagement element. Thus, in the case of a mechanical joint, the mounting region 12 and the engagement element 16 may be angularly adjusted through a series of preset angular orientations, e.g., in one degree increments, or may be limited to two preset orientations (e.g., a substantially linear orientation and an angled orientation). The ultimate angle of the longitudinal axis of the mounting region 12 (which is generally axially aligned with the longitudinal axis of the tensioning device once mounted with respect thereto) and the axis perpendicular to the engagement face of the engagement element 16 is generally between about 15° and 35°, although alternative angular orientations that fall outside the noted range may be implemented without departing from the spirit or scope of the present disclosure. The angular orientation of the mounting region (and the tensioning device) and the engagement element is generally selected so as to provide a line of ingress for instrumentation/implants and/or a line of vision to a desired anatomical location along the axis of the tensioning device, as will be apparent to persons skilled in the art.

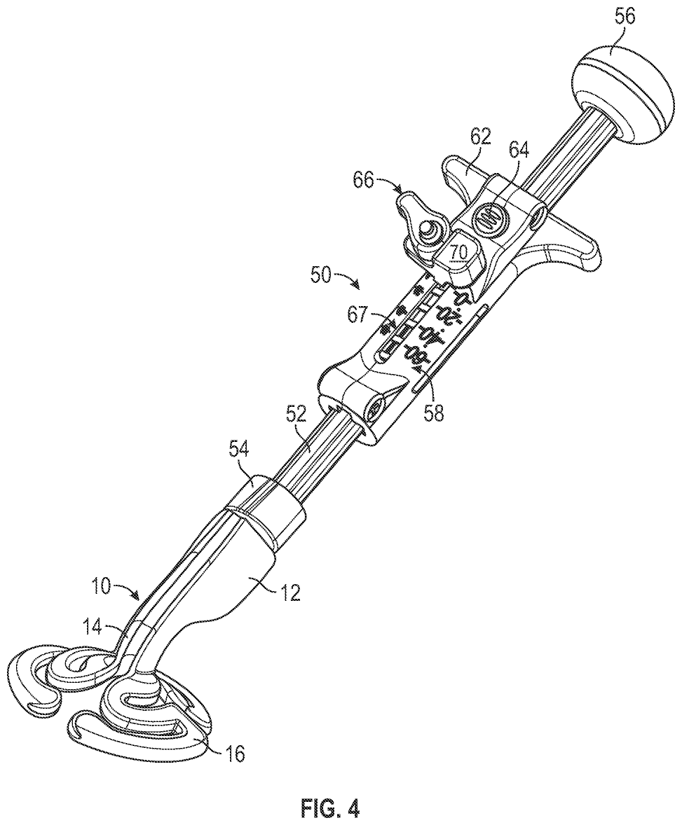

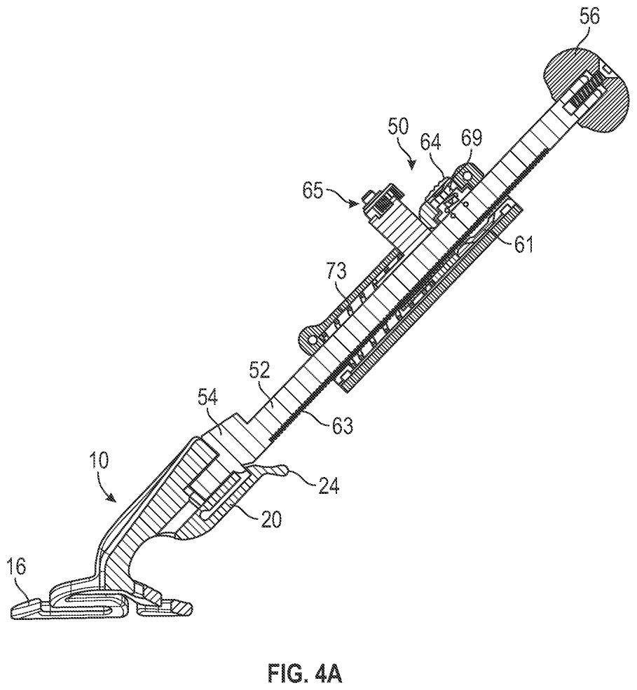

As shown in FIGS. 3, 4 and 4A, exemplary tensioning device 50 includes an elongated shaft 52, a stop 54 formed or mounted with respect to shaft 52, and a proximal handle 56 formed or mounted with respect to shaft 52. The elongated shaft 52 defines a keyed extension 60 that extends distally of stop 54. The keyed extension 60 is adapted to engage with a keyed opening 18 defined in the mounting region 12 of engagement assembly 10. As noted previously, the geometry and/or overall design of the mounting assembly is not limited to the exemplary geometry/design of tensioning device 50 and engagement assembly 10. Rather, various geometries and attachment mechanisms may be employed without departing from the spirit or scope of the present disclosure.

A slidable tensioning assembly 58 is mounted with respect to elongated shaft 52. Gripping extensions 62 are formed at (or adjacent) a proximal end of tensioning assembly 58 to permit a surgeon/practitioner to slide/translate the tensioning assembly 58 proximally relative to proximal handle 56. A locking mechanism is generally operative relative to tensioning assembly 58 and may be used to releasably secure the tensioning assembly 58 at a desired position relative to shaft 52. In the exemplary embodiment of FIGS. 3, 4 and 4A, the locking mechanism includes a button 64 that is biased by spring 69 and extends upwardly relative to tensioning assembly 58. By pressing button 64 against the bias of spring 69, a pawl 61 is disengaged from teeth associated with an inner rack 63 defined by tensioning assembly 58, thereby freeing the tensioning assembly 58 for axial movement relative to shaft 52. Upon release of button 64, the bias of spring 69 returns the button to its outward rest position and causes pawl 61 to re-engage with the teeth of inner rack 63, thereby detachably fixing the position of tensioning assembly 58 relative to shaft 52.

In the exemplary embodiment of FIGS. 3, 4 and 4A a gripping mechanism 65 for engaging with a suture associated with an implant/graft is provided that includes a spring-loaded lever arm 66 that is rotatable relative to central pin 68. Extension surface 70 may be engaged by a user while interacting with lever arm 66. Lever arm 66 includes or interacts with an internal camming mechanism that bears against a suture that is drawn within tensioning assembly 58 and through a suture passage region. Slot 72 permits the lever arm 66 and associated mechanism to travel axially relative to tensioning assembly 58. The spring that biases the lever arm/camming mechanism (e.g., a coil spring) biases the camming mechanism into securing engagement with a suture within suture passage region. Based on the orientation of the spring bias, it is possible to pull the suture proximally without manual rotation of lever arm 66. However, in order to release the suture for distal travel through the suture passage region, it is generally necessary to manually rotate lever arm 66 against the spring bias, thereby releasing engagement of the suture by the cam mechanism and allowing free travel of the suture through the suture passage region. Thus, based on the spring bias of the camming mechanism, the suture may be drawn proximally without manual release of the camming mechanism, but distal travel of the suture requires manual release of the camming mechanism (by rotation of lever arm 66). The surface of the camming mechanism that engages with the suture may be advantageously roughened to provide enhanced engagement between the camming mechanism and the suture.

A tensioning gauge 67 is associated with tensioning assembly 58. Tensioning gauge 67 includes an internal spring (not pictured) that is biased against proximal motion of tensioning assembly 58 relative to shaft 52. As a surgeon/practitioner draws the tensioning assembly 58 proximally (through interaction with gripping extensions 62), the internal spring is loaded. The degree to which the spring is loaded is correlated with the indicia on the face of tensioning assembly 58 (e.g., 0, 20N, 40N, 60N). An indicator 71 is visible through side slot 72 which allows the surgeon/practitioner to gauge the degree to which a ligament/tendon/graft is being tensioned by the disclosed assembly. A complementary indicator/side slot combination is generally provided on the opposite side of tensioning assembly (not visible in FIG. 3). In the exemplary orientation schematically depicted in FIG. 3, indicator 71 is aligned with the “0” indicia, thereby indicating that the implant/graft is not under tension. As tension is applied to the implant/graft, the indicator 71 is repositioned within side slot 73 and allowing the user to assess the level of tension being applied. The foregoing mechanism constitutes an exemplary means for measuring tension applied to a graft/implant by the slidable tensioning assembly according to the present disclosure.

With reference to FIGS. 4, 5 and 7, engagement assembly 10 is shown detachably secured relative to tensioning device 50. Thus, keyed extension 60 is positioned within the keyed opening 18 of engagement assembly 10, and latching hook 22 of latch arm 20 is engaged with a latching shelf 76 formed in stop 54. In the exemplary embodiment of FIGS. 4, 5 and 7, the proximal face of mounting region 12 abuts stop 54, thereby providing further stability to the interaction between engagement assembly 10 and tensioning device 50. The present disclosure is not limited by or to embodiments wherein the mounting region 12 abuts a structure associated with a tensioning device (e.g., stop 54). When assembled, the longitudinal axis of shaft 52 substantially aligns with the longitudinal axis defined by the mounting region 12 of engagement assembly 10 of engagement assembly 10. However, the contour region 14 angles relative to the longitudinal axes of shaft 52 and mounting region 12. As a result, an engagement plane defined by engagement element 16 is not perpendicular to the longitudinal axes of shaft 52 and/or mounting region 12, but instead is substantially perpendicular to the axis of the contour region 14 in the connection region between the contour region 14 and engagement element 16 of engagement assembly 10.

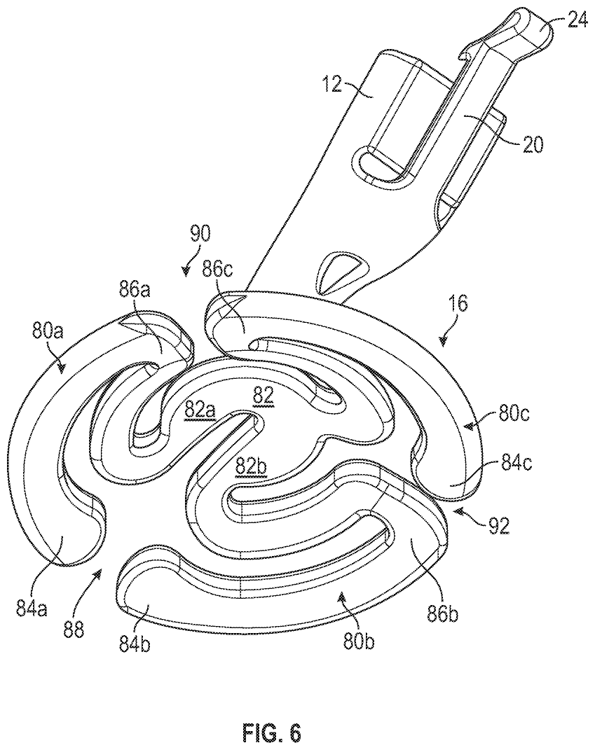

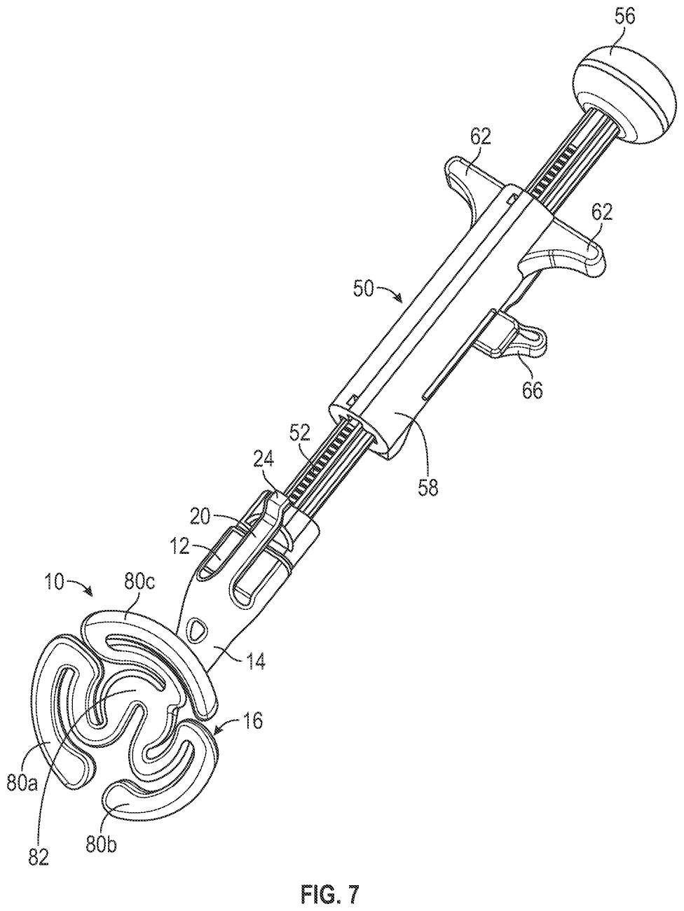

With reference to FIGS. 6 and 7, the structural design and geometry of engagement element 10 is described in greater detail. Engagement element 10 includes three “S-shaped” arms 80a, 80b, 80c that extend from a U-shaped central hub 82. Contour region 14 of engagement element 10 is joined to the central hub 82 of engagement element 16. Arm 80a extends from a first leg 82a of hub 82 and arm 80b extends from a second leg 82b of hub 82. Arm 80c extends from an arcuate region of hub 82. S-shaped arms 80a and 80b are mirror images of each other, and arm 80c is oriented in the same manner as one of the other arms, in this case arm 80b. The termination ends 84a, 84b of arms 80a, 80b are in abutting, spaced alignment with each other. Termination end 84c of arm 80c is in spaced relationship to a U-shaped elbow region 86b of arm 80b. U-shaped elbow regions 86a, 86c are in spaced relationship to each other. Thus, the overall design of engagement element 10 forms three gaps between the S-shaped arms: namely, gap 88 between the termination ends 84a, 84b of arms 80a, 80b; gap 90 between elbows 86a, 86c of arms 80a, 80c; and gap 92 between termination end 84c of arm 80c and elbow 86b of arm 80b. Although the size of gaps 88, 90 and 92 need not be equal, such gaps are generally roughly/substantially equivalent to each other (e.g., about 0.25 inch).

As is most apparent in FIGS. 1, 4 and 5, the S-shaped arms 80a, 80b, 80c extend both radially outward relative to hub 82, but also extend distally relative to hub 82. Thus, as each S-shaped arm 80 extends away from hub 82 in a substantially horizontal manner, it also extends distally relative to hub 82. As a result, when viewed from the side (as shown in FIG. 5), engagement element 10 defines a dome-shaped structure such that hub 82 is raised relative to the termination ends 84a, 84b, 84c of arms 80a, 80b, 80c. The dome-shaped geometry of engagement element 16 provides an advantageous force absorption functionality as the engagement element 16 is brought into contact with a surface because, depending on the forces involved, the engagement element 16 can flatten out (in whole or in part) to absorb forces that would otherwise be imparted to the surface. Based on the materials used in fabricating engagement element 16, the dome-shaped geometry is generally resilient such that, when the engagement element 16 is brought into contact with an anatomical surface and a downward force is applied, the dome-shaped geometry flattens. Conversely, when the engagement element 16 is removed from the anatomical site and/or the downward force removed, the dome-shaped geometry of the engagement element 16 is reestablished, i.e., the engagement element 16 resiliently returns to (or very near to) its original shape.

The flexibility of the engagement element 16 in absorbing forces, as described above, minimizes the potential for bruising/trauma/contusions to tissues surrounding an anatomical target region. In achieving the desired flexibility and resilience, the engagement element 16 is generally fabricated from a material selected from various polymers or metals with an appropriate modulus of elasticity, e.g., polymers such as acetal (polyoxymethylene), nylon, polypropylene and high molecular weight polyethylene (HMWPE), and metals such as titanium and Nitinol (nickel/titanium alloy). In exemplary implementations, e.g., where a metal is employed, an over-mold of silicon or other material having softer/less rigid properties may be undertaken to minimize traumatic effects associated with tissue engagement. The cross-section of arms 80a, 80b, 80c is generally in the range of 1/32 to ¼ inches. The cross-section of the arms themselves may take various forms, e.g., circular, oval, elliptical, rectangular, etc. As is apparent from FIGS. 4 and 6, the termination ends 84a, 84b, 84c may be enlarged relative to the remainder of arms 80a, 80b, 80c, thereby providing greater surface area for engagement with tissue in such regions, although such geometric property is not a necessity to realize the benefits of the disclosed engagement assembly 16.

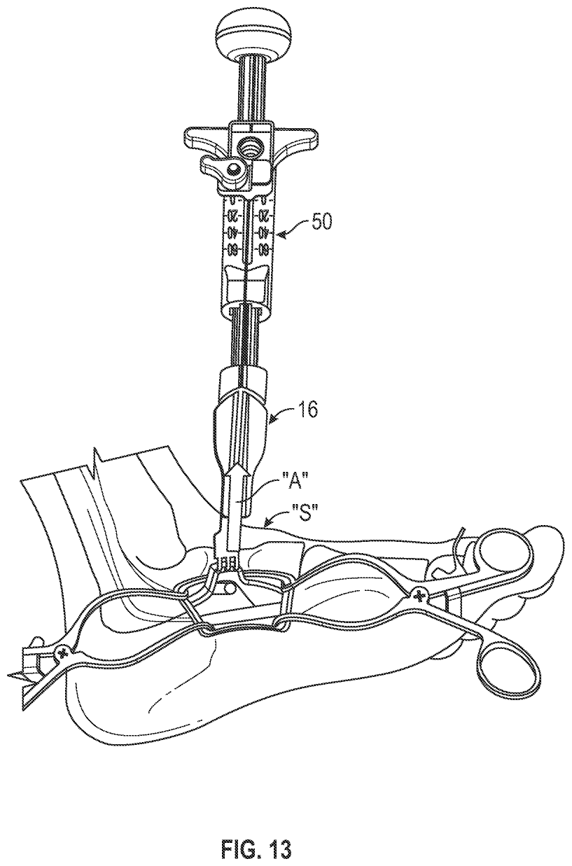

In use, the disclosed engagement assembly 16 is detachably secured relative to tensioning device 50 by introducing keyed extension 60 into keyed opening 18 and bringing latching hook 22 into engagement with latching shelf 76 in stop 54. The engagement element 16 is placed against a tissue/anatomical surface “S” adjacent a desired surgical site, as shown in FIG. 13. Ends of the replacement ligament/tendon/graft are drawn from the surgical site in the direction of arrow “A” using suture and the suture is then secured relative to tensioning device 50, i.e., by passing the suture through a suture passage region and engaging the suture within the suture passage region, e.g., with a gripping mechanism 65 as described above with reference to FIG. 3. Of note, the suture may be threaded through the end of the implant/graft/tendon/ligament in various patterns as are known to surgeons and the free end of the suture is then used to position the implant/graft/tendon/ligament into a clinically desired location. The free end of the suture is drawn into the tensioning device, as described above. The apparatus/systems of the present disclosure may be used in procedures that allow the tension fixation device to be inserted thru the prepared anatomy on either side of the tunnel channel (i.e., the side of the tensioning device or the opposite side of the tensioning device).

The surgeon/practitioner tensions the ligament/tendon/graft by sliding the tensioning assembly 58 proximally relative to shaft 52 (through interaction with gripping extensions 62). Once the desired tension is achieved (as measured by the tensioning gauge 66), the tensioning assembly 58 may be locked relative to the shaft 52, thereby maintaining the desired tension on the ligament/tendon/graft. Of note, application of the tensioning force necessarily imparts a force on the tissue adjacent the target site as the engagement element 16 is pressed thereagainst. The advantageous design/geometry of the disclosed engagement element 16 spreads such force out across the surface of arms 80a, 80b, 80c and absorbs a portion of the force through flattening of the dome-shaped geometry. In this way, potential bruising/trauma/contusion of such tissue is advantageously reduced.

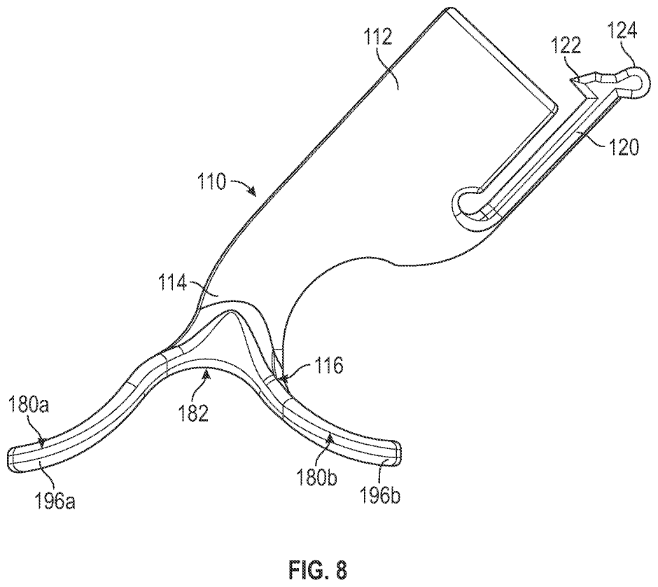

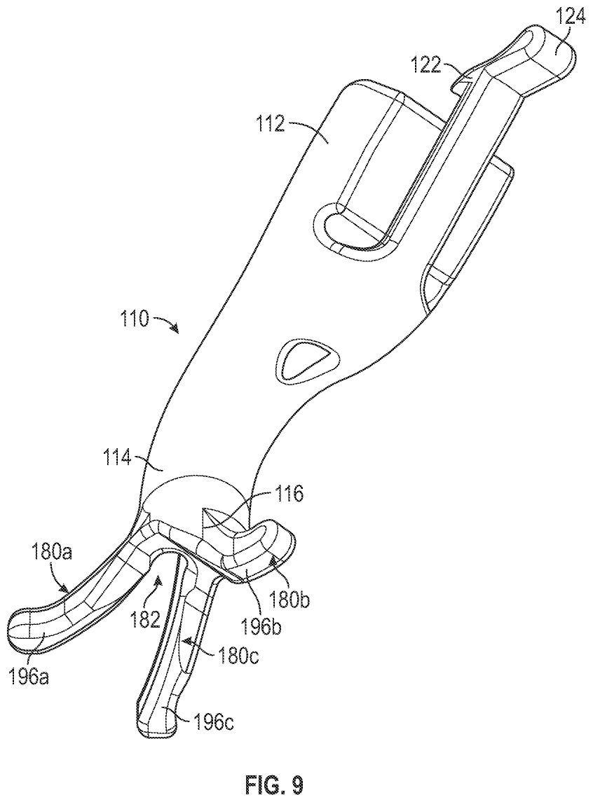

Turning to FIGS. 8 and 9, an alternative engagement assembly 110 is depicted. As with engagement assembly 10, engagement assembly 110 includes a mounting region 112, a contoured region 114 and an engagement element 116. However, the design/geometry of engagement element 116 is different from the design/geometry of engagement element 16. The engagement assembly 110 is adapted to detachably mount to a tensioning assembly, e.g., tensioning assembly 50, in the same manner as engagement assembly 10. Thus, engagement assembly 110 includes a latch arm 120 that defines a latching hook 122 and a release nub 124. A mounting feature, e.g., a keyed opening (not pictured), is formed in the mounting region to cooperate with a cooperative mounting structure associated with the tensioning assembly.

In contrast to the three S-shaped arms associated with engagement element 16, the engagement assembly 116 includes three legs 180a, 180b, 180c that together define a tripod structure. The three legs 180a, 180b, 180c extend downward and outward from a central hub region 182 that is joined relative to the contour region 114. Each of the legs defines a curved geometry such that an abutment surface 196a, 196b, 196c is formed by each leg at a distance removed from the hub region 182. The legs 180a, 180b, 180c are sufficiently flexible/resilient that, as downward force is applied from the tensioning device (i.e., as the ligament/tendon/graft is tensioned), the legs flatten, thereby absorbing and spreading such force over the underlying tissue. In this way, engagement element reduces the potential for bruising/trauma/contusion to the tissue. Once the force is removed, the legs 180a, 180b, 180c advantageously return to (or nearly to) their initial tripod orientation. The engagement element 116 is generally fabricated from a material selected from various polymers or metals with an appropriate modulus of elasticity, e.g., polymers such as acetal (polyoxymethylene), nylon, polypropylene and high molecular weight polyethylene (HMWPE), and metals such as titanium and Nitinol (nickel/titanium alloy). In exemplary implementations, e.g., where a metal is employed, an over-mold of silicon or other material having softer/less rigid properties may be undertaken to minimize traumatic effects associated with tissue engagement. The cross-section of arms 180a, 180b, 180c is generally in the range of 1/32 to ¼ inches. The cross-section of the arms themselves may take various forms, e.g., circular, oval, elliptical, rectangular, etc.

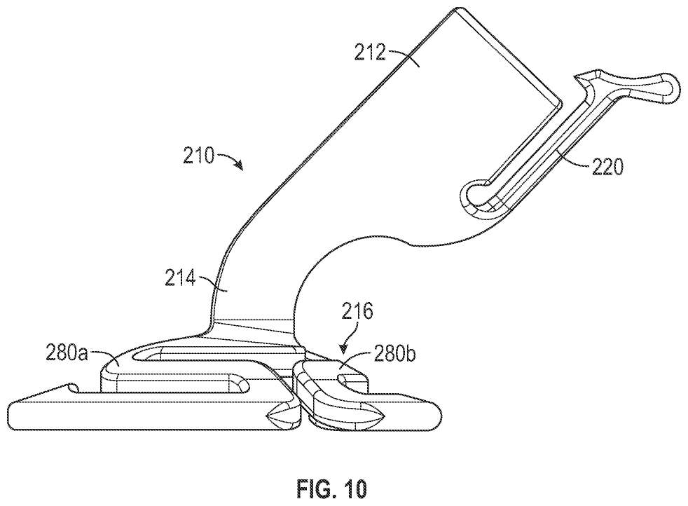

Turning to FIG. 10, a further exemplary engagement assembly 210 is depicted according to the present disclosure. Engagement assembly 210 includes mounting region 212, contour region 214, engagement element 216 and latch arm 220. The engagement element 216 is similar in design/geometry to the engagement element 16 described with reference to engagement assembly 10, except that the radially outward portions of the arms 280a, 280b, 280c (not pictured) are squared off rather than curved. Thus, the first/second/third elbow regions may feature a curved geometry or a squared geometry. In all other respects, engagement assembly 210 is the same as engagement assembly 10.

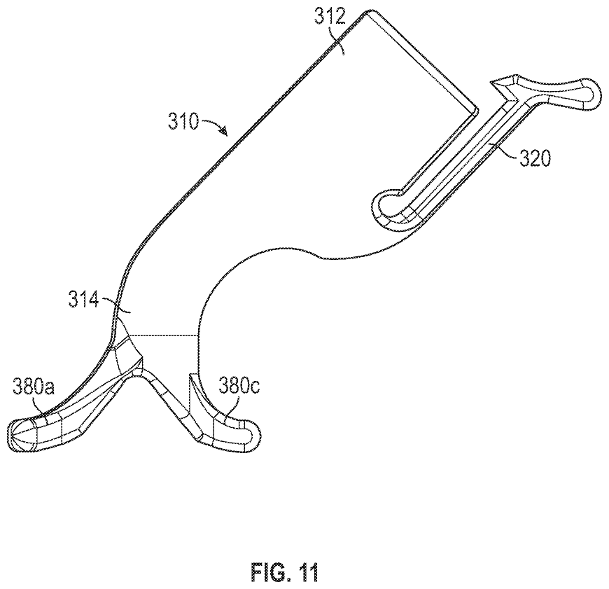

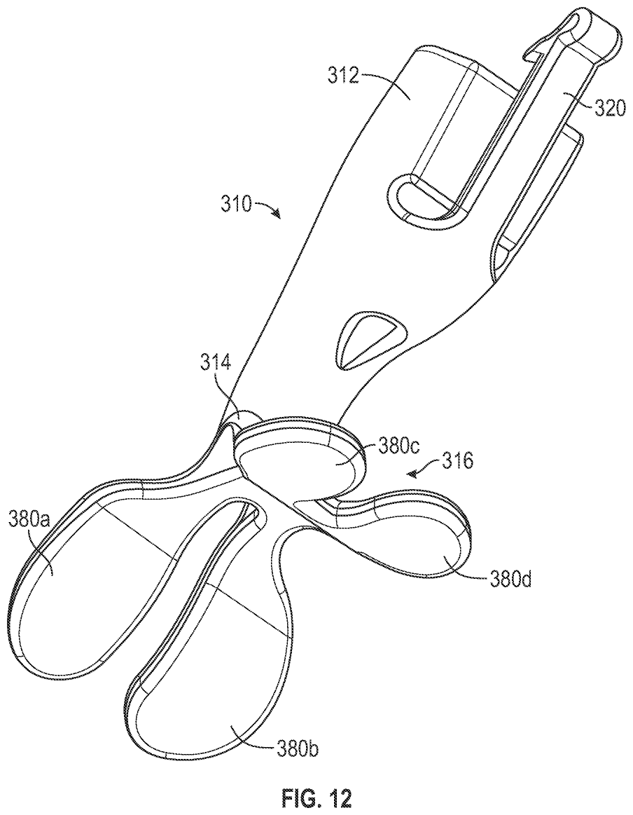

With reference to FIGS. 11 and 12, a further exemplary engagement assembly 310 is depicted according to the present disclosure. Engagement assembly 310 includes mounting region 312, contour region 314, engagement element 316 and latch arm 320. The engagement element 316 is similar in design/geometry to the engagement element 116 described with reference to engagement assembly 110, except that the three tripod legs 180a, 180b, 180c are replaced by two pairs of legs 380a, 380b and 380c, 380d that define paddle-shaped tissue abutment regions. Of note, the length of legs 380a, 380b is greater than the length of legs 380c, 380d. The relationship between the pairs of legs may be varied/adjusted in achieving the desired results, e.g., the paddle widths may be varied, the paddle thickness may be varied, the spacing of paddle regions may be varied, etc. Beyond the introduction of the two pairs of legs 380a, 380b and 380c, 380d, engagement assembly 310 is the same as engagement assembly 110 in all other respects.

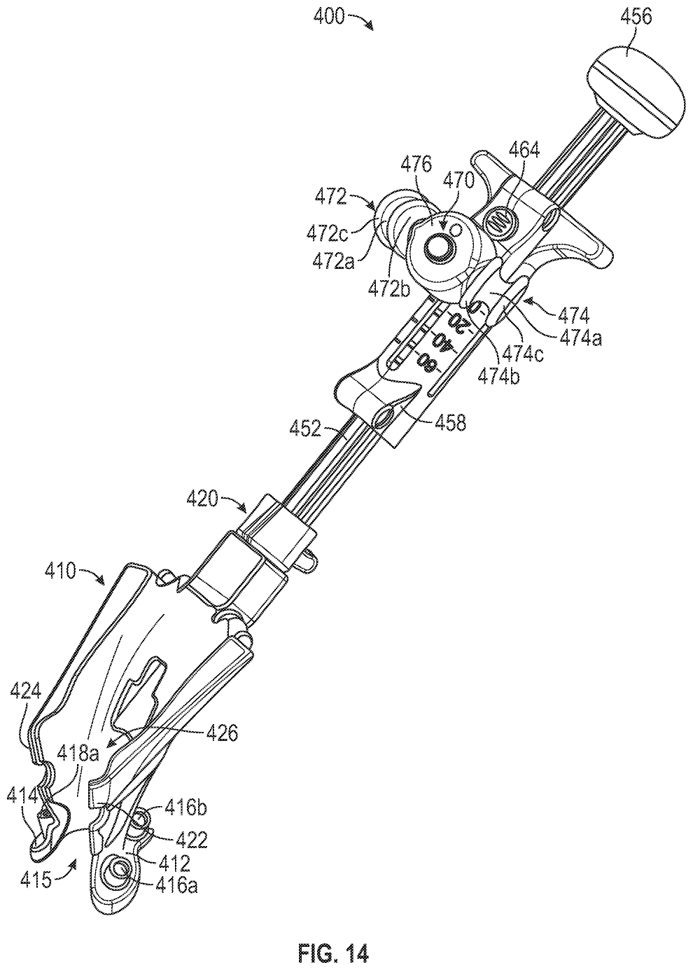

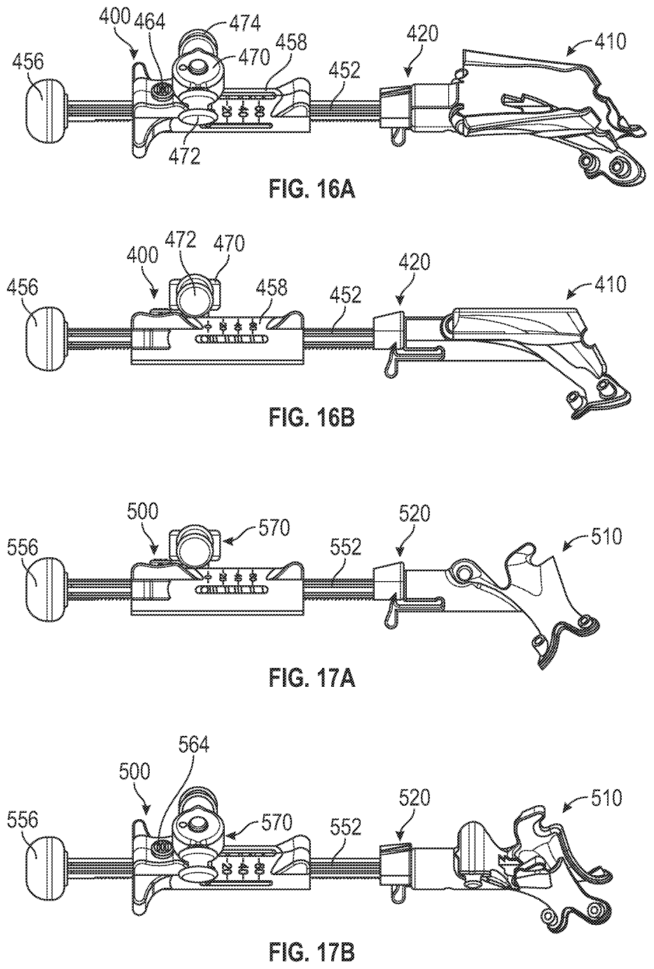

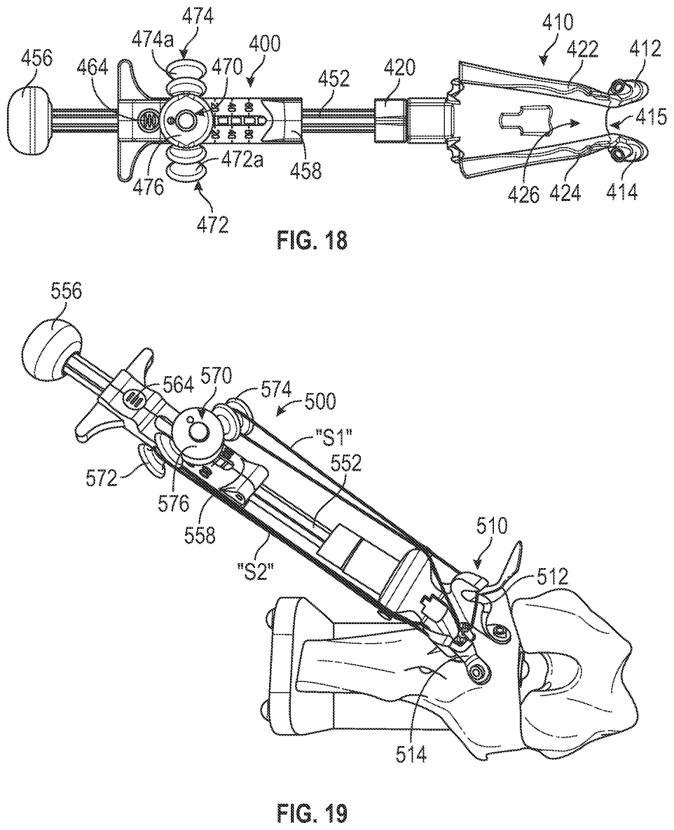

Turning to FIGS. 14, 16A, 16B and 18, an alternative graft tensioning device/engagement assembly for use in alternative implant procedures, e.g., ACL replacement procedures, is schematically depicted. Graft tensioning device 400 is detachably secured relative to engagement assembly 410. The engagement mechanism 420 includes structures and functions as described above, e.g., with reference to FIGS. 1 and 2. The graft tensioning device 400 includes an elongated shaft 452, a proximal handle 456 and a slidable tensioning assembly 458. A locking mechanism is provided that includes a spring-biased button 464 that operates to disengage a pawl from engagement with an inner rack associated with the tensioning assembly 458, as described above with reference to FIGS. 4 and 4A.

Graft tensioning device 400 includes an upstanding suture retention structure 470 that is mounted relative to the slidable tensioning assembly 458. Suture retention structure 470 includes a pair of spaced suture channels 472, 474 on opposite sides of a central support 476. Suture channels 472, 474 define central regions 472a, 474a that are bounded by inner side walls and outer side walls. Central regions 472a, 474a generally define a circular suture engagement surface that facilitates wrapping of a suture therearound. Suture channels 472, 474 may be angularly oriented relative to central support 476, such that an upper region of suture channels 472, 474 is further removed from central support 476 as compared to a lower region of suture channels 472, 474. The angular orientation may be on the order of 5° to 10° relative to a non-angular orientation.

Suture retention structure 470 is generally rotatable relative to the axis of central support 476. In this way, suture channel 472 and suture channel 474 may assume a position relative to a patient that balances the forces exerted by suture wrapped around suture channels 472, 474, respectively. Rotatable or swivel motion of retention structure 470 is generally permitted by providing a rotatable mounting mechanism (not pictured) between the retention structure 470 and the tensioning assembly 458, e.g., a ball bearing mechanism, a rotatable coupling mechanism, and the like. The rotatable mounting mechanism may include one or more stops that limit rotation of retention structure within a desired angular range, e.g., less than 30° relative to an axis that is perpendicular to shaft 452, less than 15° relative to the noted perpendicular axis, etc.

As noted above, engagement assembly 410 is detachably mounted relative to graft tensioning device 400. Engagement assembly 410 includes first and second tissue engagement surfaces 412, 414 on opposite sides of central channel region 415. Of note, central channel region 415 defines an open space between tissue engagement surfaces 412, 414 within which an implant region may be positioned in clinical use. Each of the tissue engagement surfaces 412, 414 include a plurality of spaced apertures for receipt of securing elements 416a, 416b, 418a, 418b, e.g., screws, for detachably securing the engagement assembly 410 relative to a desired anatomical location. Opposed guide arms 422, 424 are defined by engagement assembly 410 and may be used for routing sutures in a spaced/controlled manner from the implant region to the suture retention structure 470.

Engagement assembly 410 includes a contour region 426 that defines an arcuate transition for the central channel region 415. As with the previously described contour regions, contour region 426 generally defines an overall angular orientation between the axis of the graft tensioning device 400 and the plane defined by the implant region of about 15° and 35°, although alternative angular orientations that fall outside the noted range may be implemented without departing from the spirit or scope of the present disclosure.

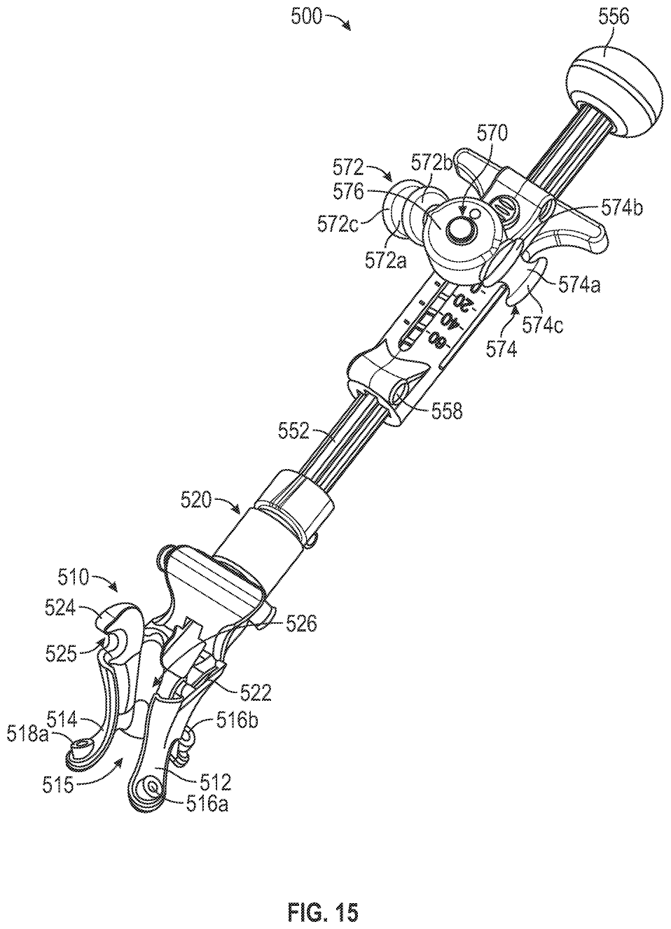

Turning to FIGS. 15, 17A and 17B, a further alternative graft tensioning device/engagement assembly for use in alternative implant procedures, e.g., ACL replacement procedures, is schematically depicted. Graft tensioning device 500 is detachably secured relative to engagement assembly 510. The engagement assembly 510 includes structures and functions as described above, e.g., with reference to FIGS. 1 and 2. The graft tensioning device 500 includes an elongated shaft 552, a proximal handle 556 and a slidable tensioning assembly 558. A locking mechanism is provided that includes a spring-biased button 564 that operates to disengage a pawl from engagement with an inner rack associated with the tensioning assembly 558, as described above with reference to FIGS. 4 and 4A.

Graft tensioning device 500 includes upstanding suture retention structure 570 (that corresponds to suture retentions structure 470 described above) that is mounted relative to the slidable tensioning assembly 558. Suture retention structure 570 includes a pair of spaced suture channels 572, 574 on opposite sides of a central support 576. Suture channels 572, 574 define central regions 572a, 574a that are bounded by inner side walls 572b, 574b and outer side walls 572c, 574c. Central regions 572a, 574a generally define a circular suture engagement surface that facilitates wrapping of a suture therearound. Suture channels 572, 574 may be angularly oriented relative to central support 576, such that an upper region of suture channels 572, 574 is further removed from central support 576 as compared to a lower region of suture channels 572, 574. The angular orientation may be on the order of 5° to 10° relative to a non-angular orientation.

Suture retention structure 570 is generally rotatable relative to the axis of central support 576. In this way, suture channel 572 and suture channel 574 may assume a position relative to a patient that balances the forces exerted by suture wrapped around suture channels 572, 574, respectively. Rotatable or swivel motion of retention structure 570 is generally permitted by providing a rotatable mounting mechanism (not pictured) between the retention structure 570 and the tensioning assembly 558, e.g., a ball bearing mechanism, a rotatable coupling mechanism, and the like. The rotatable mounting mechanism may include one or more stops that limit rotation of retention structure within a desired angular range, e.g., less than 30° relative to an axis that is perpendicular to shaft 552, less than 15° relative to the noted perpendicular axis, etc.

As noted above, engagement assembly 510 is detachably mounted relative to graft tensioning device 500. Engagement assembly 510 includes first and second tissue engagement surfaces 512, 514 on opposite sides of central channel region 515. Central channel region 515 defines an open space between tissue engagement surfaces 512, 514 within which an implant region may be positioned in clinical use. Each of the tissue engagement surfaces 512, 514 include a plurality of spaced apertures for receipt of securing elements 516a, 516b, 518a, 518b, e.g., screws, for detachably securing the engagement assembly 510 relative to a desired anatomical location. Engagement assembly 510 includes a first guide arm 522 on a first side of central channel region 515 and a second guide arm 524 on an opposite side of central channel region 515. The second guide arm 524 defines a hook region 525 for controlled engagement of suture that passes therethrough. Thus, first and second guide arms 522, 524 may be used for routing sutures in a spaced/controlled manner from the implant region to the suture retention structure 470.

As shown in FIG. 19, sutures S1 and S2 engage with retention structure 570 and engagement assembly 510 to apply balanced tension to the system.

Engagement assembly 510 includes a contour region 526 that defines an arcuate transition for the central channel region 515. As with the previously described contour regions, contour region 526 generally defines an overall angular orientation between the axis of the graft tensioning device 500 and the plane defined by the implant region of about 15° and 35°, although alternative angular orientations that fall outside the noted range may be implemented without departing from the spirit or scope of the present disclosure.

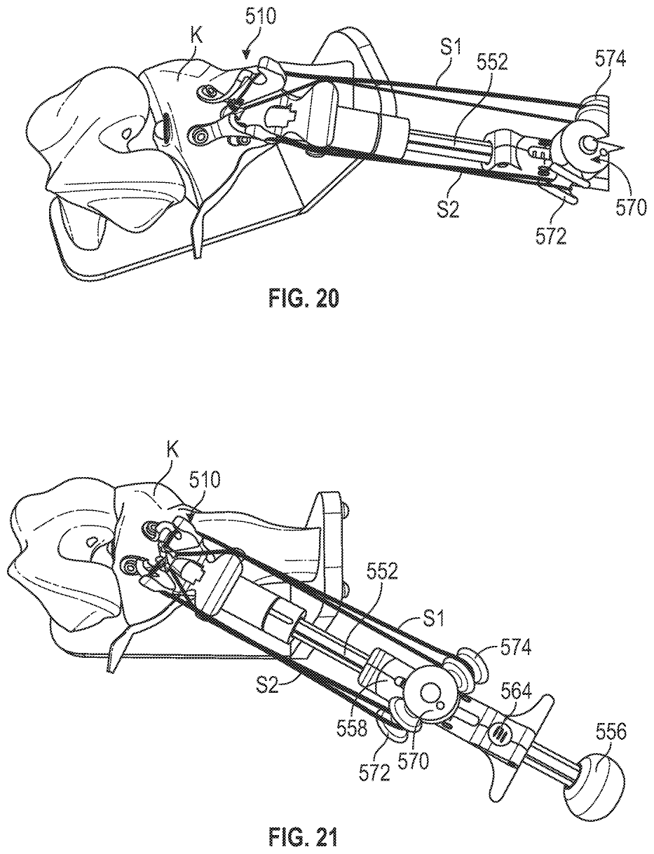

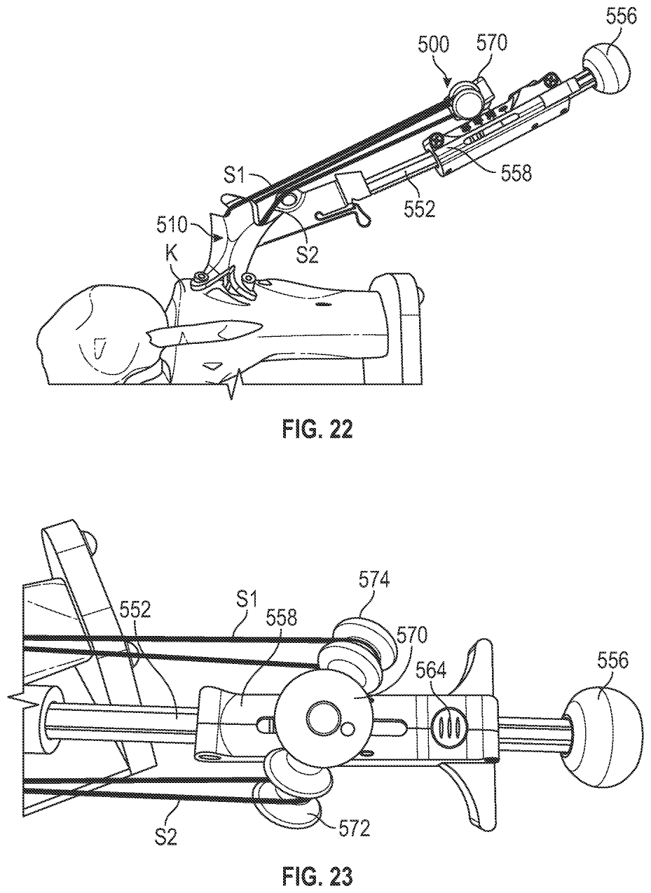

FIGS. 19-23 show various views of a graft tensioning device 500 and engagement assembly 510 mounted with respect to a knee “K” (figures employ “model” of portion of knee). The engagement assembly 510 is detachably fixed relative to the knee by four screws. The central channel region of the engagement assembly 510 opens to an implant region. The sutures that are secured relative to the implant (S1 and S2) are guided by the first and second guide arms (including the hook region defined by one of the guide arms) proximally to the suture retention structure that is rotatably mounted relative to the slidable tensioning assembly. As most visible in FIG. 23, the suture retention structure is free to rotate/swivel so as to balance the forces applied sutures secured on either side thereof. In this way, a balanced force may be applied to the implant as it is positioned relative to the surgical site.

Of note, the various engagement assemblies disclosed herein may be used interchangeably. Indeed, a surgeon/practitioner may substitute one of the disclosed engagement assemblies for another of the disclosed engagement assemblies at any time, e.g., during a surgical procedure, if desired. Thus, if the anatomical properties of a particular procedure are better suited to the design/geometry of engagement assembly 10, the surgeon/practitioner can easily detach/remove a previously selected engagement assembly, e.g., engagement assembly 110 or engagement assembly 210 or engagement assembly 310, and substitute engagement assembly 10 therefore. In addition, an engagement assembly that is particularly suited for an implant in the foot may be replaced by an engagement assembly that is particularly suited for an implant in the knee, thereby further enhancing the flexibility and modularity of the disclosed devices and systems. The disclosed modular substitutability of engagement assemblies thus greatly enhances the efficacy of the disclosed devices, systems and methods.

Although the present disclosure has been described with reference to exemplary embodiments, the present disclosure is not limited by or to such exemplary embodiments. Rather, the devices, systems and methods of the present disclosure are susceptible to various refinements, modifications and enhancements without departing from the spirit or scope of the present invention.

1. An engagement assembly for use in a surgical procedure, comprising:
a. a contoured region, and
b. an engagement element associated with a distal end of the contoured region;
wherein the engagement element is substantially dome-shaped or substantially tripod shaped and adapted for resilient flattening when pressed against a surface.

2. The engagement assembly of claim 1, further comprising a mounting feature associated with the contoured region or adjacent thereto, wherein the mounting feature facilitates detachable attachment relative to a device.

3. The engagement assembly of claim 2, wherein the device is a graft tensioning device.

4. The engagement assembly of claim 1, wherein the engagement element includes a central hub and a plurality of S-shaped arms that extend from the central hub.

5. (canceled)

6. The engagement assembly of claim 4, wherein two of the three S-shaped arms is defined by a leg that extends from the central hub and an arm member that extends from an associated leg.

7. The engagement assembly of claim 6, wherein a first S-shaped arm is defined by a first arm member that extends from a first leg, a second S-shaped arm is defined by a second arm member that extends from a second leg, and a third S-shaped arm is defined by a third arm member that extends from an arcuate region of the central hub.

8. The engagement assembly of claim 7, wherein the first S-shaped arm and the second S-shaped arm are mirror images of each other.

9. The engagement assembly of claim 7, wherein the first arm member defines a first termination end and the second arm member defines a second termination end, and wherein the first termination end and the second termination end are in abutting, spaced alignment with each other.

10. The engagement assembly of claim 9, wherein the first S-shaped arm defines a first elbow region between the first arm member and the first leg, the second S-shaped arm defines a second elbow region between the second arm member and the second leg, and wherein the third S-shaped arm defines a third termination end that is in spaced relationship to the first elbow region or the second elbow region.

11. (canceled)

12. (canceled)

13. (canceled)

14. (canceled)

15. The engagement assembly of claim 4, wherein the plurality of S-shaped arms extend both radially outward relative to the central hub and distally relative to the central hub.

16. The engagement assembly of claim 15, wherein the engagement element defines a dome-shaped structure based on the radial and distal extension of the plurality of S-shaped arms relative to the central hub.

17. The engagement assembly of claim 1, wherein the engagement element includes a central hub and a plurality of curved legs that extend from the central hub.

18. The engagement assembly of claim 1, wherein the engagement element includes a central hub and three legs that extend from the central hub that together define a tripod structure.

19. (canceled)

20. The engagement assembly of claim 1, wherein the engagement element includes a central hub and two pairs of legs that each define paddle-shaped tissue engagement regions.

21. (canceled)

22. A graft tensioning system, comprising:
a. an elongated shaft;
b. a slidable tensioning assembly movably mounted with respect to the elongated shaft; and
c. an engagement assembly according to any of the preceding claims, wherein the engagement assembly is mounted relative to or extends from the elongated shaft.

23. A graft tensioning system, comprising:
a. an elongated shaft;
b. a slidable tensioning assembly movably mounted with respect to the elongated shaft; and
c. a suture retention structure mounted relative to the slidable tensioning assembly;
wherein the suture retention structure is adapted to rotate or swivel relative to the slidable tensioning assembly.

24. The graft tensioning system according to claim 23, wherein the suture retention structure rotates or swivels in response to forces applied by sutures fixed relative to the suture retention structure.

25. The graft tensioning system according to claim 14 wherein the engagement assembly includes first and second guide arms on either side of a central channel that communicates with an open region, and wherein the first and second guide arms function to guide suture from the open region to the slidable tensioning assembly.

26. The graft tensioning system of claim 25, wherein at least one of the first and second guide arms defines a hook region.

27. (canceled)

28. (canceled)

29. (canceled)

30. (canceled)

31. The graft tensioning system of claim 22, wherein the slidable tensioning assembly includes means for measuring tension applied to a graft or implant by the slidable tensioning assembly.
